(19) United States
(12) Patent Application Publication
(10) Pub. No.: US 2004/0147574 A1
(43) Pub. Date: Jul. 29, 2004

(54) Isothiazole derivatives

(75) Inventor: Michael J. Munchhof, Salem, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/765,658

(22) Filed: Jan. 26, 2004

Related U.S. Application Data
(60) Provisional application No. 60/442,708, filed on Jan. 27, 2003.

(51) Int. Cl. C07D 275/02; A61K 31/42

(52) U.S. Cl. 514/376; 548/213; 514/342; 546/271.1

(57) ABSTRACT

Novel isothiazole compounds, including derivatives thereof, to pharmaceutical compositions containing them and to their medicinal use are described. The compounds of the present invention are potent inhibitors of transforming growth factor (“TGF”)-β signaling pathway. They are useful in the treatment of various TGF-related disease states including, for example, hyperproliferative disorders and fibrotic diseases.

### RELATED APPLICATIONS

This application claims benefit of priority under 35 U.S.C. § 119(e) to U.S. Provisional Application No. 60/442,708 filed Jan. 27, 2003, which is herein incorporated by reference in its entirety.

### BACKGROUND OF THE INVENTION

This invention relates to novel isothiazole derivatives, including derivatives thereof, to pharmaceutical compositions containing them and to their medicinal use. The compounds of the present invention are potent inhibitors of the transforming growth factor (“TGF”)-β signaling pathway. They are useful in the treatment of TGF-β related disease states including, for example, hyperproliferative disorders (e.g. tumors, cancer) and fibrotic diseases.

Isothiazole derivatives useful as anticancer agents are described in U.S. Pat. No. 6,235,764 and WO 99/62890.

There still exists a need in the art for compounds that inhibit the TGF-β signaling pathway. The present invention, as described below, answers such a need.

### SUMMARY OF THE INVENTION

The invention provides a compound of formula (I):

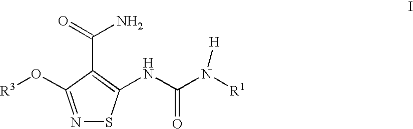

and a pharmaceutically acceptable salt, prodrug, hydrate or solvate thereof, wherein:

R¹ is (C₁-C₁₀)alkyl, (C₃-C₁₀)cycloalkyl(CH₂)ₜ—, (C₆-C₁₀)aryl(CH₂)ₜ—, or (5-10 membered heterocycle)(CH₂)ₜ—, wherein said R¹ s optionally substituted with at least one moiety selected from the group consisting of (C₁-C₆)alkyl, halo, hydroxy, (C₁-C₆)alkoxy, halo(C₁-C₆)alkoxy, oxo, and amino; preferably, R¹ is (C₁-C₁₀)alkyl; preferably, R¹ is (C₃-C₁₀)cycloalkyl(CH₂)ₜ—; preferably, R¹ is (C₆-C₁₀)aryl(CH₂)ₜ—; preferably, R¹ is (5-10 membered heterocycle)(CH₂)ₜ—;

t is an integer from 0 to 5;

R³ is (5-10 membered heteroaryl)(CH₂)ₛ—, (5-10 membered heterocycle)(CH₂)ₛ—, wherein said R³ is optionally substituted with at least one moiety selected from the group consisting of (C₁-C₆)alkyl, halo, hydroxy, (C₁-C₆)alkoxy, halo(C₁-C₆)alkoxy, oxo, and amino; and

s is an integer from 0 to 5.

Another embodiment of the invention is a compound of formula (I), as set forth above, wherein:

R¹ is as set forth above;

R³is a (2-pyridinyl)(CH₂)ₛ—, (3-pyridinyl)(CH₂)ₛ— or (4-pyridinyl)(CH₂)ₛ—;

t is an integer from 0-4; preferably, 0-3; and

s is an integer from 1-5; preferably, 1-3.

The invention also provides a compound of formula (II):

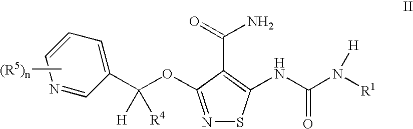

and a pharmaceutically acceptable salt, prodrug, hydrate or solvate thereof, wherein:

R¹ is (C₁-C₁₀)alkyl, (C₃-C₁₀ cycloalkyl)(CH₂)ₜ—, (C₆-C₁₀ aryl)(CH₂)ₜ—, or (5-10 membered heterocycle)(CH₂)ₜ—, wherein said R¹ is optionally substituted with at least one moiety selected from the group consisting of (C₁-C₆)alkyl, halo, hydroxy, (C₁-C₆)alkoxy, halo(C₁-C₆)alkoxy, oxo, and amino; preferably, R¹ is (C₁-C₁₀)alkyl; preferably, R¹ is (C₃-C₁₀ cycloalkyl)(CH₂)ₜ—; preferably, R¹ is (C₆-C₁₀ aryl)(CH₂)ₜ—; preferably, R¹ is (5-10 membered heterocycle)(CH₂)ₜ—;

t is an integer from 0 to 4;

R⁴ is H or (C₁-C₁₀)alkyl;

each R⁵ is independently H, (C₁-C₁₀)alkyl, (C₂-C₁₀)alkenyl, (C₂-C₁₀)alkynyl, halo, cyano, nitro, trifluoromethyl, trifluoromethoxy, azido, —OR⁶, —C(O)R⁶, —C(O)OR⁶, —NR⁷C(O)OR⁶, —OC(O)R⁶, —NR⁷SO₂R⁶, —SO₂NR⁶R⁷, —NR⁷C(O)R⁶, —C(O)NR⁶R⁷, —NR⁶R⁷, —S(O)ⱼR⁸, —SO₃H, —NR⁶ (CR⁷R⁸)ₚOR⁷, —(CH₂)_{p(C}₆-C₁₀ aryl), —SO₂(CH₂)ₚ(C₆-C₁₀)aryl, —S(CH₂)ₚ(C₆-C₁₀)aryl, —O(CH₂)ₚ(C₆-C₁₀)aryl, —(CH₂)ₚ(5-10 membered heterocyclic), and —(CR⁷R⁸)ₘOR⁷;

m is an integer from 1 to 5;

p is an integer from 0 to 5;

j is an integer from 0 to 2;

each R⁶ is independently selected from H, (C₁-C₁₀)alkyl, (C₆-C₁₀)aryl(CH₂)ₖ—, and (5-10 membered heterocyclic)(CH₂)ₖ—;

k is an integer from 0 to 5;

each R⁷ and R⁸ is independently H or (C₁-C₆)alkyl; and

n is an integer from 1 to 4.

In a preferred embodiment, the invention provides a compound of formula (II), as set forth above, selected from the group consisting of:

5-[3-(2-Cyclohex-1-enyl-ethyl)-ureido]-3-(pyridin-3-ylmethoxy)-isothiazole-4-carboxylic acid amide;

5-[3-(2,5-Dimethyl-benzyl)-ureido]-3-(pyridin-3-ylmethoxy)-isothiazole-4-carboxylic acid amide;

5-[3-(3,5-Dimethoxy-benzyl)-ureido]-3-(pyridin-3-ylmethoxy)-isothiazole-4-carboxylic acid amide;

5-[3-(2-Ethoxy-benzyl)-ureido]-3-(pyridin-3-ylmethoxy)-isothiazole-4-carboxylic acid amide;

5-{3-[2-(2-Ethoxy-phenyl)-ethyl]-ureido}-3-(pyridin-3-ylmethoxy)-isothiazole-4-carboxylic acid amide;

5-{3-[2-(3,4-Dimethoxy-phenyl)-ethyl]-ureido}-3-(pyridin-3-ylmethoxy)-isothiazole-4-carboxylic acid amide;

5-(3-Phenethyl-ureido)-3-(pyridin-3-ylmethoxy)-isothiazole-4-carboxylic acid amide;

5-{3-[2-(3-Ethoxy-4-methoxy-phenyl)-ethyl]-ureido}-3-(pyridin-3-ylmethoxy)-isothiazole-4-carboxylic acid amide;

5-{3-[2-(4-Ethoxy-phenyl)-ethyl]-ureido}-3-(pyridin-3-ylmethoxy)-isothiazole-4-carboxylic acid amide;

5-{3-[2-(4-Chloro-phenyl)-ethyl]-ureido}-3-(pyridin-3-ylmethoxy)-isothiazole-4-carboxylic acid amide;

5-{3-[2-(3-Chloro-phenyl)-ethyl]-ureido}-3-(pyridin-3-ylmethoxy)-isothiazole-4-carboxylic acid amide;

5-{3-[2-(3-Methoxy-phenyl)-ethyl]-ureido}-3-(pyridin-3-ylmethoxy)-isothiazole-4-carboxylic acid amide;

5-{3-[2-(4-Methoxy-phenyl)-ethyl]-ureido}-3-(pyridin-3-ylmethoxy)-isothiazole-4-carboxylic acid amide;

5-{3-[2-(3-Bromo-4-methoxy-phenyl)-ethyl]-ureido}-3-(pyridin-3-ylmethoxy)-isothiazole-4-carboxylic acid amide;

5-{3-[2-(4-Bromo-phenyl)-ethyl]-ureido}-3-(pyridin-3-ylmethoxy)-isothiazole-4-carboxylic acid amide;

5-{3-[2-(2-Chloro-phenyl)-ethyl]-ureido}-3-(pyridin-3-ylmethoxy)-isothiazole-4-carboxylic acid amide;

5-{3-[2-(3-Chloro-phenyl)-ethyl]-ureido}-3-(pyridin-3-ylmethoxy)-isothiazole-4-carboxylic acid amide;

5-{3-[2-(2-Fluoro-phenyl)-ethyl]-ureido}-3-(pyridin-3-ylmethoxy)-isothiazole-4-carboxylic acid amide;

5-{3-[2-(3-Fluoro-phenyl)-ethyl]-ureido}-3-(pyridin-3-ylmethoxy)-isothiazole-4-carboxylic acid amide;

5-{3-[2-(4-Fluoro-phenyl)-ethyl]-ureido}-3-(pyridin-3-ylmethoxy)-isothiazole-4-carboxylic acid amide;

5-{3-[2-(4-Ethoxy-3-methoxy-phenyl)-ethyl]-ureido}-3-(pyridin-3-ylmethoxy)-isothiazole-4-carboxylic acid amide;

5-{3-[2-(3-Ethoxy-4-methoxy-phenyl)-ethyl]-ureido}-3-(pyridin-3-ylmethoxy)-isothiazole-4-carboxylic acid amide;

5-{3-[2-(2,5-Dimethoxy-phenyl)-ethyl]-ureido}-3-(pyridin-3-ylmethoxy)-isothiazole-4-carboxylic acid amide;

5-{3-[2-(3-Methoxy-phenyl)-ethyl]-ureido}-3-(pyridin-3-ylmethoxy)-isothiazole-4-carboxylic acid amide;

5-[3-(2-Difluoromethoxy-benzyl)-ureido]-3-(pyridin-3-ylmethoxy)-isothiazole-4-carboxylic acid amide;

5-[3-(2,6-Dimethoxy-benzyl)-ureido]-3-(pyridin-3-ylmethoxy)-isothiazole-4-carboxylic acid amide;

5-[3-(2,5-Dichloro-benzyl)-ureido]-3-(pyridin-3-ylmethoxy)-isothiazole-4-carboxylic acid amide;

5-[3-(3-Morpholin-4-yl-propyl)-ureido]-3-(pyridin-3-ylmethoxy)-isothiazole-4-carboxylic acid amide; 5-[3-(2-Morpholin-4-yl-ethyl)-ureido]-3-(pyridin-3-ylmethoxy)-isothiazole-4-carboxylic acid amide;

5-[3-(2-Diethylamino-ethyl)-ureido]-3-(pyridin-3-ylmethoxy)-isothiazole-4-carboxylic acid amide;

5-[3-(3-Dimethylamino-propyl)-ureido]-3-(pyridin-3-ylmethoxy)-isothiazole-4-carboxylic acid amide;

5-{3-[2-(1-Methyl-pyrrolidin-2-yl)-ethyl]-ureido}-3-(pyridin-3-ylmethoxy)-isothiazole-4-carboxylic acid amide;

5-{3-[3-(2-Methyl-piperidin-1-yl)-propyl]-ureido}-3-(pyridin-3-ylmethoxy)-isothiazole-4-carboxylic acid amide;

(R),(R)-5-[3-(2-Hydroxy-cycloheptylmethyl)-ureido]-3-(pyridin-3-ylmethoxy)-isothiazole-4-carboxylic acid amide;

(R),(R)-5-[3-(2-Hydroxy-cyclooctylmethyl)-ureido]-3-(pyridin-3-ylmethoxy)-isothiazole-4-carboxylic acid amide;

5-[3-(2-Hydroxy-ethyl)-ureido]-3-(pyridin-3-ylmethoxy)-isothiazole-4-carboxylic acid amide;

5-[3-(2-Hydroxy-butyl)-ureido]-3-(pyridin-3-ylmethoxy)-isothiazole-4-carboxylic acid amide;

5-{3-[3-(2-Oxo-pyrrolidin-1-yl)-propyl]-ureido}-3-(pyridin-3-ylmethoxy)-isothiazole-4-carboxylic acid amide;

5-[3-(3-Imidazol-1-yl-propyl)-ureido]-3-(pyridin-3-ylmethoxy)-isothiazole-4-carboxylic acid amide;

5-(3-Benzyl-ureido)-3-(pyridin-3-ylmethoxy)-isothiazole-4-carboxylic acid amide;

5-[3-(2,5-Difluoro-benzyl)-ureido]-3-(pyridin-3-ylmethoxy)-isothiazole-4-carboxylic acid amide;

3-(1-Pyridin-3-yl-ethoxy)-5-(3-pyridin-2-ylmethyl-ureido)-isothiazole-4-carboxylic acid amide;

5-[3-(2,6-Dimethoxy-benzyl)-ureido]-3-(1-pyridin-3-yl-ethoxy)-isothiazole-4-carboxylic acid amide;

5-(3-Cyclopropylmethyl-ureido)-3-(pyridin-3-ylmethoxy)-isothiazole-4-carboxylic acid amide;

5-(3-Methyl-ureido)-3-(pyridin-3-ylmethoxy)-isothiazole-4-carboxylic acid amide;

5-(3-Methyl-ureido)-3-(1-pyridin-3-yl-ethoxy)-isothiazole-4-carboxylic acid amide; and

5-[3-(3,5-Dichloro-benzyl)-ureido]-3-(pyridin-3-ylmethoxy)-isothiazole-4-carboxylic acid amide.

The invention provides a pharmaceutical composition comprising a compound of the invention and a pharmaceutically acceptable carrier, each as set forth herein.

The invention provides a method of treating a TGF-related disease state in a mammal comprising the step of administering to the mammal suffering from the TGF-related disease state a therapeutically effective amount of a compound of the invention, each as set forth herein. In a preferred embodiment of the invention, the TGF-related disease state is selected from the group consisting of hyperproliferative disorders and fibrotic diseases. Examples of a hyperproliferative disorder include, but are not limited to, a tumor and cancer. Examples of a fibrotic disease include, but are not limited to glomerulonephritis, diabetic nephropathy, hepatic fibrosis, pulmonary fibrosis, intimal hyperplasia and restenosis, scleroderma, and dermal scarring.

A compound of the invention can be used in the manufacture of a medicament for the therapeutic treatment of a TGF-related disease state in a mammal, each as described herein.

### DEFINITIONS

As used herein, the article “a” and “an” refers to both the singular and plural form of the object to which it refers unless indicated otherwise.

As used herein, the term “alkyl,” as well as the alkyl moieties of other groups referred to herein (e.g., alkoxy) refers to a linear or branched, saturated hydrocarbon (e.g., methyl, ethyl, n-propyl, isopropyl, n-butyl, isobutyl, secondary-butyl, tertiary-butyl).

As used herein, the term “cycloalkyl” refers to a mono- or bicyclic carbocyclic ring (e.g., cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1 ]octanyl and bicyclo[5.2.0]nonanyl).

As used herein, the term “halogen” or “halo” refers to fluoro, chloro, bromo or iodo or fluoride, chloride, bromide or iodide.

As used herein, the term “halo-substituted alkyl” or “haloalkyl” refers to an alkyl radical, as set forth above, substituted with one or more halogens, as set forth above. Examples include, but are not limited to, chloromethyl, dichloromethyl, fluoromethyl, difluoromethyl, trifluoromethyl, and 2,2,2-trichloroethyl.

As used herein, the term “alkenyl” refers to a linear or branched hydrocarbon chain radical containing at least two carbon atoms and at least one double bond. Examples include, but are not limited to, ethenyl, 1-propenyl, 2-propenyl (allyl), iso-propenyl, 2-methyl-1-propenyl, 1-butenyl, and 2-butenyl.

As used herein, the term “alkynyl” refers to a linear or branched hydrocarbon chain radical containing at least one triple bond. Examples include, but are not limited to, ethynyl, propynyl, and butynyl.

As used herein, the term “alkoxy” refers to an “—O-alkyl” moiety where “alkyl” is as defined above.

As used herein, the term “carbonyl” refers to a “>C═O” moiety. Alkoxycarbonylamino (i.e., alkoxy(C═O)—NH—) refers to an alkyl carbamate group. The carbonyl group is also equivalently defined herein as (C═O). As used herein, the term “aryl” refers to an aromatic radical such as, for example, phenyl, naphthyl, tetrahydronaphthyl, and indanyl.

As used herein, the term “heteroaryl” refers to an aromatic group containing at least one heteroatom selected from O, S and N. Preferably, the term “heteroaryl” refers to a 5- to 10-membered aromatic group containing at least one heteroatom selected from 0, S and N. For example, heteroaryl groups include, but are not limited to, pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, and indolyl.

As used herein, the term “heterocycle”, “heterocyclic” or “heterocyclyl” refers to a saturated, unsaturated or aromatic C₃-C₂₀ mono-, bi- or polycyclic group containing at least one heteroatom selected from N, O, and S. Preferably, the term “heterocycle”, “heterocyclic” or “heterocyclyl” refers to a 5- to 10-membered ring system containing at least one heteroatom selected from N, O, and S. Examples of heterocyclic groups include, but are not limited to, azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxcithiazinyl, indolinyl, isoindolinyl, quincuclidinyl, chromanyl, isochromanyl, benzocazinyl, and the like. Examples of monocyclic saturated or unsaturated ring systems are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholin-yl, 1,2-tetrahydrothiazin-2-yl, 1,3-tetrahydrothiazin-3-yl, tetrahydrothiadiazin-yl, morpholin-yl, 1,2-tetrahydrodiazin-2-yl, 1,3-tetrahydrodiazin-1-yl, 1,4-oxazin-2-yl, and 1,2,5-oxathiazin-4-yl.

The term “oxo” refers to a double bonded oxygen moiety, i.e., ═O.

As used herein, the term “pharmaceutically acceptable acid addition salt” refers to non-toxic acid addition salts, i.e., salts derived from pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate)] salts.

As used herein, the term “pharmaceutically acceptable base addition salt” refers to non-toxic base addition salts, i.e., salts derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

As used herein, the term “suitable substituent”, “substituent” or “substituted” refers to a chemically and pharmaceutically acceptable functional group, i.e., a moiety that does not negate the therapeutic activity of the inventive compounds. Such suitable substituents may be routinely selected by those skilled in the art. Illustrative examples of suitable substituents include, but are not limited to, carbonyl, halo, haloalkyl, perfluoroalkyl, perfluoroalkoxy, alkyl, alkenyl, alkynyl, hydroxy, oxo, mercapto, alkylthio, alkoxy, aryl or heteroaryl, aryloxy or heteroaryloxy, aralkyl or heteroaralkyl, aralkoxy or heteroaralkoxy, HO—(C═O)—, ester, amido, ether, amino, alkyl- and dialkylamino, cyano, nitro, carbamoyl, alkylcarbonyl, alkoxycarbonyl, alkylaminocarbonyl, dialkylaminocarbonyl, arylcarbonyl, aryloxycarbonyl, alkylsulfonyl, arylsulfonyl and the like. Those skilled in the art will appreciate that many substituents can be substituted by additional substituents.

As used herein, the term “TGF-related disease state” refers to any disease state mediated by the production of TGF-β.

As used herein, the term “hyperproliferative disorder” refers to any disorder resulting from an abnormally high rate of cell division which results in a rapid proliferation of the cells.

### DETAILED DESCRIPTION OF THE INVENTION

A compound of the invention can be readily prepared by following the procedures outlined in the schemes illustrated below and typical synthetic procedures familiar to those skilled in the art. It is to be understood that the scope of the invention is not limited in any way by the scope of the following examples and preparations.

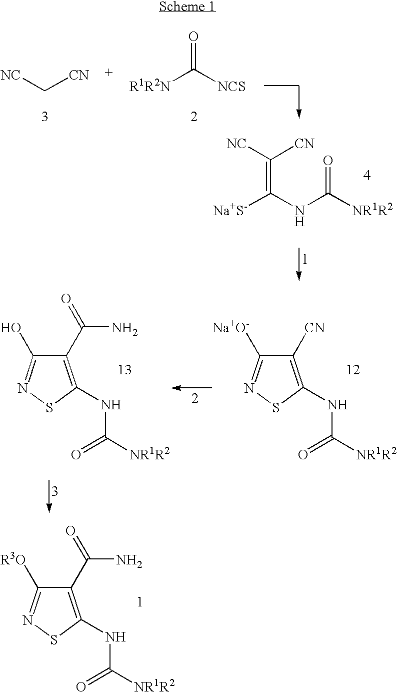

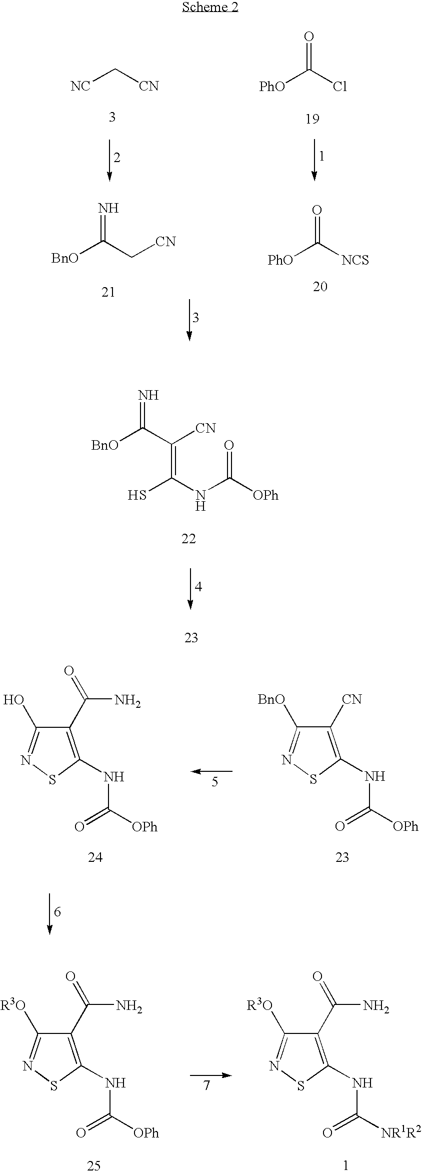

Scheme 1 illustrates a method of preparing a compound 1. The starting compound of formula 4 was prepared by treating malonitrile 3 and isocyanate 2 (R¹ and R² are not H but otherwise are as defined above) with a suitably strong base, such as an alkoxide base, preferably sodium ethoxide, in a protic solvent, such as an alcohol, preferably ethanol, at a temperature ranging from −20° C. to 50° C., preferably 0° C. to 25° C., over a period of about 12 to 24 hours. Next, in step 1 of Scheme 1, a solution of the salt of formula 4 in an inert solvent containing water or, preferably, in water alone, was treated with an oxidizing reagent, preferably dihydrogen peroxide. The mixture was held at a temperature and time sufficient to effect dissolution and cyclization, preferably at reflux for about 15 minutes, and then cooled to provide the compound of formula 12. In step 2 of Scheme 1, the compound of formula 12 was added to an acid solution, preferably concentrated sulfuric acid, followed by water sufficient to effect hydration, preferably about 10 equivalents, and was stirred at a temperature ranging from −20° C. and 100° C., preferably ambient temperature, for a period to effect hydration, preferably overnight. The mixture was then treated with water or, preferably, ice to provide the compound of formula 13. In step 3 of Scheme 1, the compound of formula 13 was treated with a base, preferably potassium tert-butoxide, in an inert solvent, preferably DMF, at a temperature ranging from −78° C. to 100° C., preferably ambient temperature. To this mixture was added an R³ containing electrophile, such as an R³ containing alkyl halide or sulfonate, preferably an iodide or bromide of such compound. The mixture was stirred until the reaction was complete as judged by thin layer chromatography (TLC) analysis to provide a compound 1.

Scheme 2 illustrates another method of preparing a compound 1. In step 1 of Scheme 2, a mixture of a thiocyanate salt, preferably potassium thiocyanate, in an inert solvent, preferably ethyl acetate, was stirred, preferably vigorously, under an inert atmosphere, overnight to powder the salt. This mixture was then treated with an aryl chloroformate of the formula 19 (Ph is phenyl) and the resulting mixture was stirred at a temperature ranging from −40° C. to ambient temperature, preferably about 5° C., for a period sufficient to effect reaction, preferably about 8 hours. The solid byproduct was filtered off and the product was kept cool, preferably not above ambient temperature. The product was redissolved in a suitable inert solvent, preferably ether, and additional insoluble byproduct was removed. After concentration, the product was again redissolved in a suitable inert solvent, preferably hexane, and additional insoluble byproducts removed. The compound of formula 20 was then isolated. In step 2 of Scheme 2, an acidic solution, preferably ethereal HCl, was treated with the compound of formula 3. Upon dissolution, the solution was cooled, preferably to 10° C., and was treated with an alcohol, preferably benzyl alcohol. After additional stirring, the mixture was held at a given temperature, preferably about 5° C., for a period sufficient to allow complete reaction, typically about 4 days, to provide the compound of formula 21. In step 3 of Scheme 2, a solution of the compound of formula 21 in a suitable inert solvent, preferably acetonitrile, at a temperature ranging from −40° C. to ambient temperature, preferably 0° C., was treated with a solution of the compound of formula 20 in a suitable inert solvent, preferably acetonitrile. The reaction was kept at a temperature ranging from 0° C. to ambient temperature, preferably ambient temperature, to effect reaction. The mixture was then kept at a temperature appropriate to increase solidification of the product, preferably about 5° C., for period sufficient to maximize yield, preferably about 2 days. The compound of formula 22 (Bn is benzyl) was then isolated. In step 4 of Scheme 2, the compound of formula 22 was taken up in a suitable inert solvent, preferably acetonitrile, at a temperature ranging from −40° C. and 40° C., preferably 0° C., and treated with a base, preferably pyridine, and an oxidant, preferably a solution of bromine or iodine in a suitable inert solvent, preferably acetonitrile. The mixture was then stirred at a temperature sufficient to effect reaction, preferably at 0° C. for about 1 hour followed by another hour at ambient temperature. The mixture was then allowed to stand at a temperature sufficient to increase solidification, preferably at 5° C., for a sufficient period, preferably overnight. The compound of formula 23 was then isolated. In step 5 of Scheme 2, the hydration and deprotection of the compound of formula 23 was effected by treatment with an acid, preferably concentrated sulfuric acid. If the compound of formula 23 was sufficiently wet with water from the previous step, no additional water is added. If the compound of formula 23 was dry, then additional water was added, preferably about 10 equivalents. The reaction was carried out at a temperature ranging from −20° C. to 100° C., preferably ambient temperature, for a period sufficient to effect complete reaction, typically marked by complete dissolution and preferably about 3 hours. After the reaction was completed, additional sulfuric acid was added to achieve complete conversion. The mixture was then treated with water or, preferably, ice. The compound of formula 24 was then isolated. In step 6 of Scheme 2, the compound of formula 24 was combined with a trivalent phosphine, preferably triphenyl phosphine, and an R³ containing alcohol, and was treated with an azodicarboxylate derivative, preferably diisopropyl azodicarboxylate, and stirring was continued for a period of at least 1 minute. The compound of formula 25 was then isolated. In step 7 of Scheme 2, a mixture of the compound of formula 25 in a suitable inert solvent, preferably THF, was treated with a desired amine of the formula R¹R²NH and kept at a temperature sufficient to effect reaction, typically 0° C. to 100° C., preferably 50° C. to 70° C., for a period ranging from 1 hour to 48 hours, preferably overnight. A compound 1 was then isolated.

All pharmaceutically acceptable salts, prodrugs, hydrates and solvates of a compound of the invention are also encompassed by the invention.

A compound of the invention that is basic in nature is capable of forming a wide variety of different salts with various inorganic and organic acids. Although such salts must be pharmaceutically acceptable for administration to a mammal, it is often desirable in practice to initially isolate a compound of the invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

A compound of the invention that is acidic in nature is capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the invention. Such non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

An isotopically-labelled derivative of a compound of the invention is within the scope of this invention. According to the invention, an isotopically-labelled derivative is identical to the corresponding compound of the invention but for the fact that one or more atoms are replaced by an atom having an atomic mass or mass number different from the atomic mass or mass number usually found in nature. Examples of isotopes that can be incorporated into a compound of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Certain isotopically-labelled compounds of the invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. An isotopically labelled compound of the invention can be prepared using means known in the art. In general, an isotopically labeled compound of the invention, may be prepared by substituting a readily available isotopically labelled reagent for a non-isotopically labelled reagent.

Prodrugs of a compound of the invention are also encompassed. For instance, free carboxyl groups can be derivatized as amides or alkyl esters. The amide and ester moieties may incorporate groups including but not limited to ether, amine and carboxylic acid functionalities. Free hydroxy groups may be derivatized using groups including but not limited to hemisuccinates, phosphate esters, dimethylaminoacetates, and phosphoryloxymethyloxy-carbonyls, as outlined in D. Fleisher, R. Bong, B. H. Stewart, Advanced Drug Delivery Reviews (1996) (I)9, 115. Carbamate prodrugs of hydroxy and amino groups are also included, as are carbonate prodrugs and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in R. P. Robinson et al., J. Medicinal Chemistry (1996) 39, 10. An amino acid residue, or a polypeptide chain of two or more (e.g., two, three or four) amino acid residues is covalently joined through an amide or ester bond to a free amino, hydroxy or carboxylic acid group of a compound of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

A compound of the invention may have an asymmetric or chiral center and therefore exist in different enantiomeric or diasteromeric forms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomer mixtures and pure enantiomers are considered as part of the invention.

This invention relates to the use of all optical isomers and stereoisomers of a compound of the invention and mixtures thereof. A compound of the invention may also exist as tautomers. This invention relates to the use of all such tautomers and mixtures thereof.

The present invention also provides a pharmaceutical composition containing at least one compound of the invention and at least one pharmaceutically acceptable carrier. The pharmaceutically acceptable carrier may be any such carrier known in the art including those described in, for example, Remington's Pharmaceutical Sciences, Mack Publishing Co., (A. R. Gennaro edit. 1985). A pharmaceutical composition of the invention may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the compound of the invention therein may be combined with various sweetening or flavoring agents, coloring matters or dyes and, if desired, emulsifying agents or suspending agents, together with diluents such as water, ethanol, propylene glycol, glycerin, or combinations thereof.

A pharmaceutical composition of the invention may be prepared by conventional means known in the art including, for example, mixing at least one compound of the invention with a pharmaceutically acceptable carrier.

A pharmaceutical composition of the invention may be used in the treatment of a TGF-related disease state or hyperproliferative disorder, each as described herein, in a mammal. Thus, a compound of the invention may be formulated as a pharmaceutical composition for administration by any method that enables delivery of the compound to the site of action including, for example, oral, topical, buccal, intranasal, parenteral (e.g., intravenous, intramuscular, intravascular, infusion or subcutaneous), intraduodenal or rectal administration or in a form suitable for administration by inhalation or insufflation.

For oral administration, the pharmaceutical composition may take the form of, for example, a tablet, capsule, or pill prepared by conventional means with a pharmaceutically acceptable excipient such as a binding agent (e.g., pregelatinized maize starch, polyvinylpyrrolidone or hydroxypropyl methylcellulose); filler (e.g., lactose, microcrystalline cellulose or calcium phosphate); lubricant (e.g., magnesium stearate, talc or silica); disintegrant (e.g., potato starch or sodium starch glycolate); or wetting agent (e.g., sodium lauryl sulphate). The tablets may be coated by methods well known in the art. Liquid preparations for oral administration may take the form of a, for example, solution, syrup or suspension, or they may be presented as a dry product for constitution with water or other suitable vehicle before use. Such liquid preparations may be prepared by conventional means with a pharmaceutically acceptable additive such as a suspending agent (e.g., sorbitol syrup, methyl cellulose or hydrogenated edible fats); emulsifying agent (e.g., lecithin or acacia); non-aqueous vehicle (e.g., almond oil, oily esters or ethyl alcohol); and preservative (e.g., methyl or propyl p-hydroxybenzoates or sorbic acid).

For buccal administration, the composition may take the form of tablets or lozenges formulated in conventional manner.

A compound of the present invention may also be formulated for sustained release delivery according to methods well known to those of ordinary skill in the art. Examples of such formulations can be found in U.S. Pat. Nos. 3,538,214, 4,060,598, 4,173,626, 3,119,742, and 3,492,397, which are herein incorporated by reference in their entirety.

A compound of the invention may be formulated for parenteral administration by injection, including using conventional catheterization techniques or infusion. Formulations for injection may be presented in unit dosage form, e.g., in ampules or in multi-dose containers, with an added preservative. Such dosage forms can be suitably buffered, if desired. The compositions may take such forms as suspensions, solutions or emulsions in oily or aqueous vehicles, and may contain a formulating agent such as a suspending, stabilizing and/or dispersing agent. Alternatively, the active ingredient may be in powder form for reconstitution with a suitable vehicle, e.g., sterile pyrogen-free water, before use.

A compound of the invention may also be formulated in rectal compositions such as suppositories or retention enemas, e.g., containing conventional suppository bases such as cocoa butter or other glycerides.

For topical administration, a compound of the invention may be formulated as an ointment or cream.

For intranasal administration or administration by inhalation, a compound of the invention may be conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the compound of the invention. Capsules and cartridges (made, for example, from gelatin) for use in an inhaler or insufflator may be formulated containing a powder mix of a compound of the invention and a suitable powder base such as lactose or starch.

A proposed dose of a compound of the invention for oral, parenteral or buccal administration to the average adult human for the treatment of a TGF-related disease state is about 0.1 mg to about 2000 mg, preferably, about 0.1 mg to about 200 mg of the active ingredient per unit dose which could be administered, for example, 1 to 4 times per day.

Aerosol formulations for treatment of the conditions referred to above in the average adult human are preferably arranged so that each metered dose or “puff” of aerosol contains about 20 μg to about 10,000μ, preferably, about 20 μg to about 1000 μg of a compound of the invention. The overall daily dose with an aerosol will be within the range about 100 μg to about 100 mg, preferably, about 100 μg to about 10 mg. Administration may be several times daily, for example 2, 3, 4 or 8 times, giving for example, 1, 2 or 3 doses each time.

Aerosol combination formulations for treatment of the conditions referred to above in the average adult human are preferably arranged so that each metered dose or “puff” of aerosol contains from about 0.01 mg to about 1000 mg, preferably, about 0.01 mg to about 100 mg of a compound of this invention, more preferably from about 1 mg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 20,000 mg , preferably, about 0.01 mg to about 2000 mg of a compound of the invention, more preferably from about 1 mg to about 200 mg. Administration may be several times daily, for example 2, 3, 4 or 8 times, giving for example, 1, 2 or 3 doses each time.

This invention also encompasses pharmaceutical compositions containing as well as methods of treatment comprising administering a prodrug of at least one compound of the invention. As used herein, the term “prodrug” refers to a pharmacologically inactive derivative of a parent drug molecule that requires biotransformation, either spontaneous or enzymatic, within the organism to release the active drug. Prodrugs are variations or derivatives of the compounds of this invention which have groups cleavable under metabolic conditions. Prodrugs become the compounds of the invention which are pharmaceutically active in vivo, when they undergo solvolysis under physiological conditions or undergo enzymatic degradation. Prodrug compounds of this invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992). Prodrugs commonly known in the art include acid derivatives well known to practitioners of the art, such as, for example, esters prepared by reaction of the parent acids with a suitable alcohol, or amides prepared by reaction of the parent acid compound with an amine, or basic groups reacted to form an acylated base derivative. Moreover, the prodrug derivatives of this invention may be combined with other features herein taught to enhance bioavailability. For example, a compound of the invention having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of the invention. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds wherein carbonates, carbamates, amides and alkyl esters which are covalently bonded to the above substituents of a compound of the invention through the carbonyl carbon prodrug sidechain.

A compound of the invention is a potent inhibitor of transforming growth factor (“TGF”)-β signaling pathway and are therefore of use in therapy.

Accordingly, the present invention provides a method of treating a TGF-related disease in a mammal (animal or human) comprising the step of administering a therapeutically effective amount of at least one compound of the invention to the animal or human suffering from the TGF-related disease state.

As used herein, the term “therapeutically effective amount” refers to an amount of a compound of the invention required to inhibit the TGF-β signaling pathway. As would be understood by one of skill in the art, a “therapeutically effective amount” will vary from patient to patient and will be determined on a case by case basis. Factors to consider include, but are not limited to, the patient being treated, weight, health, compound administered, the severity of the disorder or condition, the rate of administration and the judgment of the prescribing physician, etc.

There are numerous disease states that can be treated by inhibition of the TGF-β signaling pathway. Such disease states include, but are not limited to, all types of hyperproliferative disorders (e.g., breast cancer, lung cancer, colon cancer, prostate cancer, ovarian cancer, pancreatic cancer, melanoma, all hematological malignancies, etc.) as well as all types of fibrotic diseases (e.g., glomerulonephritis, diabetic nephropathy, hepatic fibrosis, pulmonary fibrosis, arterial hyperplasia and restenosis, scleroderma, and dermal scarring). Other disease states that can be treated by inhibition of the TGF-β signaling pathway also include those described in U.S. Pat. No. 6,235,764.

According to the invention, in the treatment of a TGF-related disease state, a compound of the invention, as described herein, whether alone or as part of a pharmaceutical composition may be combined with another compound(s) of the invention and/or with another therapeutic agent(s). Examples of suitable therapeutic agent(s) include, but are not limited to, standard non-steroidal anti-inflammatory agents (hereinafter NSAID's) (e.g, piroxicam, diclofenac), propionic acids (e.g., naproxen, flubiprofen, fenoprofen, ketoprofen and ibuprofen), fenamates (e.g., mefenamic acid, indomethacin, sulindac, apazone), pyrazolones (e.g., phenylbutazone), salicylates (e.g., aspirin), COX-2 inhibitors (e.g., celecoxib, valdecoxib, rofecoxib and etoricoxib), analgesics and intraarticular therapies (e.g., corticosteroids) and hyaluronic acids (e.g., hyalgan and synvisc), anticancer agents (e.g., endostatin and angiostatin), cytotoxic drugs (e.g., adriamycin, daunomycin, cis-platinum, etoposide, taxol, taxotere),alkaloids (e.g., vincristine), and antimetabolites (e.g., methotrexate), cardiovascular agents (e.g., calcium channel blockers), lipid lowering agents (e.g., statins), fibrates, beta-blockers, Ace inhibitors, Angiotensin-2 receptor antagonists and platelet aggregation inhibitors, CNS agents (e.g., as antidepressants (such as sertraline)), anti-Parkinsonian drugs (e.g., deprenyl, L-dopa, Requip, Mirapex), MAOB inhibitors (e.g., selegine and rasagiline), comP inhibitors (e.g., Tasmar), A-2 inhibitors, dopamine reuptake inhibitors, NMDA antagonists, Nicotine agonists, Dopamine agonists and inhibitors of neuronal nitric oxide synthase), anti-Alzheimer's drugs (e.g., donepezil, tacrine, COX-2 inhibitors, propentofylline or metryfonate), osteoporosis agents (e.g., roloxifene, droloxifene, lasofoxifene or fosomax), immunosuppressant agents (e.g., FK-506 and rapamycin), an anti-tumour substance (e.g., mitotic inhibitors (e.g. vinblastine)), an alkylating agent (e.g., cis-platin, carboplatin and cyclophosphamide), an anti-metabolite (e.g., 5-fluorouracil, cytosine arabinoside and hydroxyurea, or one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid), a growth factor inhibitor, a cell cycle inhibitor, an intercalating antibiotic (e.g., adriamycin and bleomycin), an enzyme (e.g., interferon), and an anti-hormone such as anti-estrogen (e.g., Nolvadex® (tamoxifen)) or, an anti-androgen (e.g., Casodex® (4′-cyano-3-(4-fluorophenylsulphonyl)-2-hydroxy-2-methyl-3′-(trifluoromethyl)propionanilide)). Such conjoint treatment may be achieved by way of the simultaneous, sequential or separate dosing of the individual components of the treatment.

### Biological Activity

The activity of the compounds of the invention for the various TGF-related disease states described herein can be determined according to one or more of the following assays. According to the invention, a compound of the invention exhibits an in vitro IC₅₀ value of about 0.1 nM-100 nM.

The compounds of the present invention also possess differential activity (i.e. are selective for) for TβRII over TβRI and TβRIII. Selectivity is measured in standard assays as a IC₅₀ ratio of inhibition in each assay.

### TGF-β TypE II ReCeptor (TβRII) Kinase Assay Protocol

Phosphorylation of myelin basic protein (MBP) by the TβRII kinase was measured as follows: 80 microliters of MBP (Upstate Biotechnology #13-104) diluted in kinase reaction buffer (KRB) containing 50 mM MOPS, 5 mM MgCl₂, pH 7.2 to yield a final concentration of 3 micromolar MBP was added to each well of a Millipore 96-well multiscreen-DP 0.65 micron filtration plate (#MADPNOB50). 20 microliters of inhibitor diluted in KRB was added to appropriate wells to yield the desired final concentration (10-0.03 micromolar). 10 microliters of a mixture of ATP (Sigma #A-5394) and ³³P-ATP (Perkin Elmer #NEG/602H) diluted in KRB was added to yield a final concentration of 0.25 micromolar ATP and 0.02 microcuries of ³³P-ATP per well. 10 microliters of a GST-TβRII fusion protein (glutathione S-transferase at the N-terminal end of the cytoplasmic domain of TβRII-amino acids 193-567 with A to V change at 438) diluted in KRB was added to each well to yield a final concentration of 27 nanomolar GST-TβRII. Plates were mixed and incubated for 90 minutes at room temperature. After the reaction incubation, 100 microliters of cold 20% trichloroacetic acid (Aldrich #25,139-9) was added per well and plates were mixed and incubated for 60 minutes at 4° C. Liquid was then removed from the wells using a Millipore vacuum manifold. Plates were washed once with 200 microliters per well of cold 10% trichloroacetic acid followed by two washes with 100 microliters per well of cold 10% trichloroacetic acid. Plates were allowed to dry overnight at room temperature. 20 microliters of Wallac OptiPhase SuperMix scintillation cocktail was added to each well. Plates were sealed and counted using a Wallac 1450 Microbeta liquid scintillation counter. The potency of inhibitors was determined by their ability to reduce TβRII-mediated phosphorylation of the MBP substrate.

### ALK-5 (TβRI) Kinase Assay Protocol

The kinase assays were performed with 65 nM GST-ALK5 and 84 nM GST—Smad3 in 50 mM HEPES, 5 mM MgCl₂, 1 mM CaCl₂, 1 mM dithiothreitol, and 3_M ATP. Reactions were incubated with 0.5_Ci of [33 P]_ATPfor 3 h at 30° C. Phosphorylated protein was captured on P-81 paper (Whatman, Maidstone, England), washed with 0.5% phosphoric acid, and counted by liquid scintillation. Alternatively, Smad3 or Smad1 protein was also coated onto FlashPlate Sterile Basic Microplates (PerkinElmer Life Sciences, Boston, Mass.). Kinase assays were then performed in Flash-Plates with same assay conditions using either the kinase domain of ALK5 with Smad3 as substrate or the kinase domain of ALK6 (BMP receptor) with Smad1 as substrate. Plates were washed three times with phosphate buffer and counted by TopCount (Packard Bio-science, Meriden, Conn.). (Laping, N.J. et al. Molecular Pharmacology 62:58-64 (2002)).

### KDR/VEGF Receptor Assay Protocol

The in vitro activity of a compound of the invention in inhibiting the KDRNEGF receptor may be determined by the following procedure.

The ability of a compound of the invention to inhibit tyrosine kinase activity may be measured using a recombinant enzyme in an assay that measures the ability of compounds to inhibit the phosphorylation of the exogenous substrate, polyGlu Tyr (PGT, Sigma™, 4:1). The kinase domain of the human KDR/VEGF receptor (amino acids 805-1350) is expressed in Sf9 insect cells as a glutathione S-transferase (GST)-fusion protein using the baculovirus expression system. The protein is purified from the lysates of these cells using glutathione agarose affinity columns. The enzyme assay is performed in 96-well plates that are coated with the PGT substrate (0.625 μg PGT per well). Test compounds are diluted in dimethylsulfoxide (DMSO), and then added to the PGT plates so that the final concentration of DMSO in the assay is 1.6% (v/v). The recombinant enzyme is diluted in phosphorylation buffer (50 mM Hepes, pH 7.3, 125 mM NaCl, 24 mM MgCl₂). The reaction is initiated by the addition of ATP to a final concentration of 10 μM. After a 30 minute incubation at room temperature with shaking, the reaction is aspirated, and the plates are washed with wash buffer (PBS-containing 0.1% Tween-20). The amount of phosphorylated PGT is quantitated by incubation with a HRP-conjugated (HRP is horseradish peroxidase) PY-54 antibody (Transduction Labs), developed with TMB peroxidase (TMB is 3,3′,5,5′-tetramethylbenzidine), and the reaction is quantitated on a BioRad™ Microplate reader at 450 nM. Inhibition of the kinase enzymatic activity by the test compound is detected as a reduced absorbance, and the concentration of the compound that is required to inhibit the signal by 50% is reported as the IC₅₀ value for the test compound.

To measure the ability of the compounds to inhibit KDR tyrosine kinase activity for the full length protein that exists in a cellular context, the porcine aortic endothelial (PAE) cells transfected with the human KDR (Waltenberger et al., J. Biol. Chem. 269:26988, 1994) may be used. Cells are plated and allowed to attach to 96-well dishes in the same media (Ham's F12) with 10% FBS (fetal bovine serum). The cells are then washed, re-fed with serum depleted media that contains 0.1 % (v/v) bovine serum albumin (BSA), and allowed to incubate for 24 hours. Immediately prior to dosing with compound, the cells are re-fed with the serum depleted media (without BSA). Test compounds, dissolved in DMSO, are diluted into the media (final DMSO concentration 0.5% (v/v)). At the end of a 2 hour incubation, VEGF₁₆₅ (50 ng/ml final) is added to the media for an 8 minute incubation. The cells are washed and lysed in HNTG buffer (20 mM Hepes, pH 7.5, 150 mM NaCl, 0.2% Triton™ X-100, 10% glycerol, 0.2 mM PMSF (phenymethylsulfonyl fluoride), 1 μg/ml pepstatin, 1 μg/ml leupeptin, 1 μg/ml aprotonin, 2 mM sodium pyrophosphate, 2 mM sodium orthovanadate). The extent of phosphorylation of KDR is measured using an ELISA assay. The 96-well plates are coated with 1 μg per well of goat anti-rabbit antibody. Unbound antibody is washed off the plate and remaining sites are blocked with Superblock buffer (Pierce) prior to addition of the anti-flk-1 C-20 antibody (0.5 μg per plate, Santa Cruz). Any unbound antibody is washed off the plates prior to addition of the cell lysate. After a 2 hour incubation of the lysates with the flk-1 antibody, the KDR associated phosphotyrosine is quantitated by development with the HRP-conjugated PY-54 antibody and TMB, as described above. The ability of the compounds to inhibit the VEGF-stimulated autophosphorylation reaction by 50%, relative to VEGF-stimulated controls is reported as the IC₅₀ value for the test compound.

The ability of the compounds to inhibit mitogenesis in human endothelial cells is measured by their ability to inhibit ³H-thymidine incorporation into HUVE cells (human umbilical vein endothelial cells, Clonetics™). This assay has been well described in the literature (Waltenberger J et al. J. Biol. Chem. 269: 26988, 1994; Cao Y et al. J. Biol. Chem. 271: 3154, 1996). Briefly, 10⁴ cells are plated in collagen-coated 24-well plates and allowed to attach. Cells are re-fed in serum-free media, and 24 hours later are treated with various concentrations of compound (prepared in DMSO, final concentration of DMSO in the assay is 0.2% v/v), and 2-30 ng/ml VEGF₁₆₅. During the last 3 hours of the 24 hour compound treatment, the cells are pulsed with ³H thymidine (NEN, 1 μCi per well). The media are then removed, and the cells washed extensively with ice-cold Hank's balanced salt solution, and then 2 times with ice cold trichloroacetic acid (10% v/v). The cells are lysed by the addition of 0.2 ml of 0.1 N NaOH, and the lysates transferred into scintillation vials. The wells are then washed with 0.2 ml of 0.1 N HCl, and this wash is then transferred to the vials. The extent of ³H thymidine incorporation is measured by scintillation counting. The ability of the compounds to inhibit incorporation by 50%, relative to control (VEGF treatment with DMSO vehicle only) is reported as the IC₅₀ value for the test compound.

The activity of the compounds of the invention, in vivo, can be determined by the amount of inhibition of tumor growth by a test compound relative to a control. The tumor growth inhibitory effects of various compounds are measured according to the methods of Corbett T. H., et al. “Tumor Induction Relationships in Development of Transplantable Cancers of the Colon in Mice for Chemotherapy Assays, with a Note on Carcinogen Structure”, Cancer Res., 35, 2434-2439 (1975) and Corbett, T. H., et al., “A Mouse Colon-tumor Model for Experimental Therapy”, Cancer Chemother. Rep. (Part 2)”, 5, 169-186 (1975), with slight modifications. Tumors are induced in the flank by s.c. injection of 1×106 log phase cultured tumor cells suspended in 0.1-0.2 ml PBS. After sufficient time has elapsed for the tumors to become palpable (5-6 mm in diameter), the test animals (athymic mice) are treated with compound of the invention (formulated by dissolution in appropriate diluent, for example water or 5% Gelucire™ 44/14 rn PBS by the intraperitoneal (ip) or oral (po) routes of administration once or twice daily for 5-10 consecutive days. In order to determine an anti-tumor effect, the tumor is measured in millimeters with Vernier calipers across two diameters and the tumor volume (mm³) is calculated using the formula: Tumor weight=(length×[width]²)/2, according to the methods of Geran, R. I., et al. “Protocols for Screening Chemical Agents and Natural Products Against Animal Tumors and Other Biological Systems”, Third Edition, Cancer Chemother. Rep., 3, 1-104 (1972). The flank site of tumor implantation provides reproducible dose/response effects for a variety of chemotherapeutic agents, and the method of measurement (tumor diameter) is a reliable method for assessing tumor growth rates.

### EXAMPLES

### Example 1

### Mitsunobu Coupling

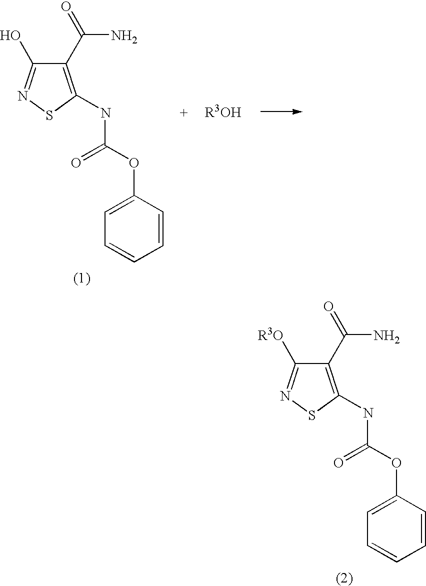

Carbamate (1) (1.0 eq) was combined with 1.0 eq alcohol R³OH, where R³ is as defined herein, and 1.5 eq. triphenylphosphine in anhydrous THF under nitrogen in a flame-dried flask. The reaction vessel was surrounded by a room temperature water bath to control any exotherm during the subsequent addition. A solution of 1.5 eq. of a 1 M solution of diethyl azodicarboxylate in anhydrous THF was slowly added dropwise over 20 minutes. Once the addition was complete, the reaction was stirred an additional 5 minutes at room temperature. The reaction mixture was quenched by addition of a small amount of methanol and concentrated to dryness. The crude residue was dissolved in chloroform and poured into water and extracted three times with chloroform. The combined organics were dried over sodium sulfate, filtered, and concentrated. The material was purified by silica gel chromatography to yield desired product (2).

### Example 2

### Urea Formation

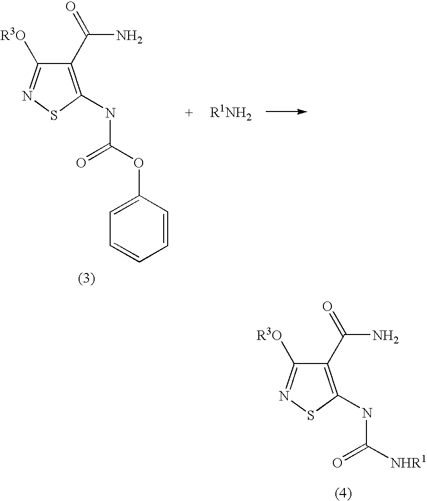

1.0 eq. of 0.2M carbamate (3) in N,N-dimethylacetamide with 3.75% N-methylmorpholine was combined with 1.5 eq. of 0.2M amine R¹NH₂ in N,N-dimethylacetamide with 3.75% N-methylmorpholine. R¹ and R³ are each as defined herein. The reaction mixture was shaken at 80° C. for 2 hours and then allowed to cool to room temperature. 10.0 eq. N-methylisatoic anhydride resin was added and the reaction shaken overnight at room temperature. Resin was removed by filtration and the crude material purified by preparative HPLC to yield desired product (4).

| | | | | LC- | |
|---|---|---|---|---|---|
| | | HPLC data | | MS | TβRII |
| Example | Compound | t_{R} = (min) | % purity | M+ | Activity uM |
| 3 | 5-[3-(2-Cyclohex-1-enyl-ethyl)-ureido]-3-(pyridin-3- | 5.55 | 99 | 402 | 1.61 |
| | ylmethoxy)-isothiazole-4-carboxylic acid amide | | | | |
| 4 | 5-[3-(2,5-Dimethyl-benzyl)-ureido]-3-(pyridin-3- | 5.57 | 100 | 412 | 1.6 |
| | ylmethoxy)-isothiazole-4-carboxylic acid amide | | | | |
| 5 | 5-[3-(3,5-Dimethoxy-benzyl)-ureido]-3-(pyridin-3- | 4.98 | 100 | 444 | 0.353 |
| | ylmethoxy)-isothiazole-4-carboxylic acid amide | | | | |
| 6 | 5-{3-[2-(3,4-Dimethoxy-phenyl)-ethyl]-ureido}-3-(pyridin- | 4.71 | 100 | 458 | 0.754 |
| | 3-ylmethoxy)-isothiazole-4-carboxylic acid amide | | | | |
| 7 | 5-(3-Phenethyl-ureido)-3-(pyridin-3-ylmethoxy)- | 5.1 | 100 | 398 | 0.94 |
| | isothiazole-4-carboxylic acid amide | | | | |
| 8 | 5-{3-[2-(3-Ethoxy-4-methoxy-phenyl)-ethyl]-ureido}-3- | 5.02 | 100 | 472 | 1.97 |
| | (pyridin-3-ylmethoxy)-isothiazole-4-carboxylic acid | | | | |
| | amide | | | | |
| 9 | 5-{3-[2-(3-Chloro-phenyl)-ethyl]-ureido}-3-(pyridin-3- | 5.56 | 100 | 432 | 2.26 |
| | ylmethoxy)-isothiazole-4-carboxylic acid amide | | | | |
| 10 | 5-{3-[2-(3-Methoxy-phenyl)-ethyl]-ureido}-3-(pyridin-3- | 5.1 | 100 | 428 | 2.12 |
| | ylmethoxy)-isothiazole-4-carboxylic acid amide | | | | |
| 11 | 5-{3-[2-(2-Chloro-phenyl)-ethyl]-ureido}-3-(pyridin-3- | 5.49 | 100 | 432 | 2.99 |
| | ylmethoxy)-isothiazole-4-carboxylic acid amide | | | | |
| 12 | 5-{3-[2-(2-Fluoro-phenyl)-ethyl]-ureido}-3-(pyridin-3- | 5.18 | 100 | 416 | 3.12 |
| | ylmethoxy)-isothiazole-4-carboxylic acid amide | | | | |
| 13 | 5-{3-[2-(3-Fluoro-phenyl)-ethyl]-ureido}-3-(pyridin-3- | 5.21 | 100 | 416 | 1.48 |
| | ylmethoxy)-isothiazole-4-carboxylic acid amide | | | | |
| 14 | 5-{3-[2-(4-Ethoxy-3-methoxy-phenyl)-ethyl]-ureido}-3- | 5.06 | 100 | 472 | 0.551 |
| | (pyridin-3-ylmethoxy)-isothiazole-4-carboxylic acid | | | | |
| | amide | | | | |
| 15 | 5-[3-(3-Morpholin-4-yl-propyl)-ureido]-3-(pyridin-3- | 2.98 | 97 | 421 | 0.437 |
| | ylmethoxy)-isothiazole-4-carboxylic acid amide | | | | |
| 16 | 5-[3-(2-Morpholin-4-yl-ethyl)-ureido]-3-(pyridin-3- | 2.99 | 97 | 407 | 0.855 |
| | ylmethoxy)-isothiazole-4-carboxylic acid amide | | | | |
| 17 | 5-[3-(2-Diethylamino-ethyl)-ureido]-3-(pyridin-3- | 3.09 | 96 | 393 | 1.94 |
| | ylmethoxy)-isothiazole-4-carboxylic acid amide | | | | |
| 18 | 5-[3-(3-Dimethylamino-propyl)-ureido]-3-(pyridin-3- | 2.92 | 96 | 379 | 0.801 |
| | ylmethoxy)-isothiazole-4-carboxylic acid amide | | | | |
| 19 | 5-{3-[2-(1-Methyl-pyrrolidin-2-yl)-ethyl]-ureido}-3-(pyridin- | 3.09 | 97 | 405 | 0.457 |
| | 3-ylmethoxy)-isothiazole-4-carboxylic acid amide | | | | |
| 20 | 5-{3-[3-(2-Methyl-piperidin-1-yl)-propyl]-ureido}-3- | 3.32 | 100 | 433 | 0.966 |
| | (pyridin-3-ylmethoxy)-isothiazole-4-carboxylic acid | | | | |
| | amide | | | | |
| 21 | (R),(R)-5-[3-(2-Hydroxy-cycloheptylmethyl)-ureido]-3- | 4.41 | 100 | 420 | 0.759 |
| | (pyridin-3-ylmethoxy)-isothiazole-4-carboxylic acid | | | | |
| | amide | | | | |
| 22 | (R),(R)-5-[3-(2-Hydroxy-cyclooctylmethyl)-ureido]-3- | 4.69 | 100 | 434 | 0.753 |
| | (pyridin-3-ylmethoxy)-isothiazole-4-carboxylic acid | | | | |
| | amide | | | | |
| 23 | 5-[3-(2-Hydroxy-ethyl)-ureido]-3-(pyridin-3-ylmethoxy)- | 3.15 | 99 | 338 | 0.349 |
| | isothiazole-4-carboxylic acid amide | | | | |
| 24 | 5-[3-(2-Hydroxy-butyl)-ureido]-3-(pyridin-3-ylmethoxy)- | 3.72 | 100 | 366 | 0.669 |
| | isothiazole-4-carboxylic acid amide | | | | |
| 25 | 3-(1-Pyridin-3-yl-ethoxy)-5-(3-pyridin-2-ylmethyl-ureido)- | 4.04 | 85 | 399 | 7.39 |
| | isothiazole-4-carboxylic acid amide | | | | |
| 26 | 5-[3-(2,6-Dimethoxy-benzyl)-ureido]-3-(1-pyridin-3-yl- | 5.27 | 100 | 458 | 0.502 |
| | ethoxy)-isothiazole-4-carboxylic acid amide | | | | |
| 27 | 5-(3-Methyl-ureido)-3-(1-pyridin-3-yl-ethoxy)-isothiazole- | 3.59 | 100 | 322 | 0.71 |
| | 4-carboxylic acid amide | | | | |

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

The claimed invention is:

1. A compound of formula (I): 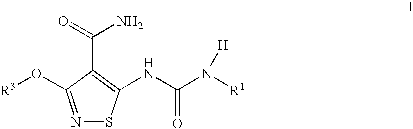
including a pharmaceutically acceptable salt, prodrug, hydrate or solvate thereof, wherein:
R¹ is (C₁-C₁₀)alkyl, (C₃-C₁₀)cycloalkyl(CH₂)ₜ—, (C₆-C₁₀)aryl(CH₂)ₜ—, or (5-10 membered heterocycle)(CH₂)ₜ—, wherein said R¹ is optionally substituted with at least one moiety selected from the group consisting of (C₁-C₆) alkyl, halo, hydroxy, (C₁-C₆)alkoxy, halo(C₁-C₆)alkoxy, oxo, and amino;
t is an integer from 0 to 5;
R³ is (5-10 membered heteroaryl)(CH₂)_{s—, (}5-10 membered heterocycle)(CH₂)ₛ—, wherein said R³ is optionally substituted with at least one moiety selected from the group consisting of (C₁-C₆)alkyl, halo, hydroxy, (C₁-C₆)alkoxy, halo(C₁-C₆)alkoxy, oxo, and amino; and
s is an integer from 0 to 5.

2. A compound of claim 1, wherein:
R³ is a (2-pyridinyl)(CH₂)ₛ—, (3-pyridinyl)(CH₂)ₛ— or (4-pyridinyl)(CH₂)ₛ—;
t is an integer from 0-4; and
s is an integer from 1-5.

3. A compound of claim 1, wherein R¹ is (C₁-C₁₀)alkyl.

4. A compound of claim 1, wherein R¹ is (C₃-C₁₀)cycloalkyl(CH₂)ₜ—.

5. A compound of claim 1, wherein R¹ is (C₆-C₁₀)aryl(CH₂)ₜ—.

6. A compound of claim 5 wherein:
R³ is a (2-pyridinyl)(CH₂)ₛ—, (3-pyridinyl)(CH₂)ₛ— or (4-pyridinyl)(CH₂)ₛ—;
t is an integer from 0-4; and
s is an integer from 1-5.

7. A compound of claim 6, wherein:
t is an integer from 0-3; and
s is an integer from 1-3.

8. A compound of claim 1, wherein R¹ is (5-10 membered heterocycle)(CH₂)ₜ—.

9. A compound of claim 8 wherein:
R³ is a (2-pyridinyl)(CH₂)ₛ—, (3-pyridinyl)(CH₂)ₛ— or (4-pyridinyl)(CH₂)ₛ—;
t is an integer from 0-4; and
s is an integer from 1-5.

10. A compound of claim 9, wherein:
t is an integer from 0-3; and
s is an integer from 1-3.

11. A compound of formula (II): 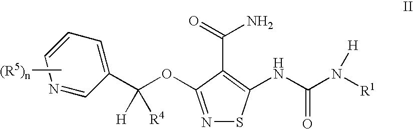
including a pharmaceutically acceptable salt, prodrug, hydrate or solvate thereof, wherein:
R₁ is C₁-C₁₀ alkyl, (C₃-C₁₀ cycloalkyl)(CH₂)ₜ—, (C₆-C₁₀ aryl)(CH₂)ₜ—, or (5-10 membered heterocycle)(CH₂)ₜ—, wherein said R¹ is optionally substituted with at least one moiety selected from the group consisting of (C₁-C₆)alkyl, halo, hydroxy, (C₁-C₆)alkoxy, halo(C₁-C₆)alkoxy, oxo, and amino;
t is an integer from 0 to 4;
R⁴ is H or (C₁-C₁₀)alkyl;
each R⁵ is independently H, (C₁-C₁₀)alkyl, (C₂-C₁₀)alkenyl, (C₂-C₁₀)alkynyl, halo, cyano, nitro, trifluoromethyl, trifluoromethoxy, azido, —OR⁶, —C(O)R⁶, —C(O)OR⁶, —NR⁷C(O)OR⁶, —OC(O)R⁶, —NR⁷SO₂R⁶, —SO₂NR⁶R⁷, —NR⁷C(O)R⁶, —C(O)NR⁶R⁷, —NR⁶R⁷, —S(O)ⱼR⁸, —SO₃H, —NR⁶ (CR⁷R⁸)ₚOR⁷, —(CH₂)ₚ(C₆-C₁₀ aryl), —SO₂(CH₂)ₚ(C₆-C₁₀)aryl, —S(CH₂)ₚ(C₆-C₁₀)aryl, —O(CH₂)ₚ(C₆-C₁₀)aryl, —(CH₂)ₚ(5-10 membered heterocyclic), and —(CR⁷R⁸)ₘOR⁷;
m is an integer from 1 to 5;
p is an integer from 0 to 5;
j is an integer from 0 to 2;
each R⁶ is independently selected from H, (C₁-C₁₀)alkyl, (C₆-C₁₀)aryl(CH₂)ₖ—, and (5-10 membered heterocyclic)(CH₂)ₖ—;
k is an integer from 0 to 5;
each R⁷ and R⁸ is independently H or (C₁-C₆)alkyl; and
n is an integer from 1 to 4.

12. A compound of claim 11 selected from the group consisting of:
5-[3-(2-Cyclohex-1-enyl-ethyl)-ureido]-3-(pyridin-3-ylmethoxy)-isothiazole-4-carboxylic acid amide;
5-[3-(2,5-Dimethyl-benzyl)-ureido]-3-(pyridin-3-ylmethoxy)-isothiazole-4-carboxylic acid amide;
5-[3-(3,5-Dimethoxy-benzyl)-ureido]-3-(pyridin-3-ylmethoxy)-isothiazole-4-carboxylic acid amide;
5-[3-(2-Ethoxy-benzyl)-ureido]-3-(pyridin-3-ylmethoxy)-isothiazole-4-carboxylic acid amide;
5-{3-[2-(2-Ethoxy-phenyl)-ethyl]-ureido}-3-(pyridin-3-ylmethoxy)-isothiazole-4-carboxylic acid amide;
5-{3-[2-(3,4-Dimethoxy-phenyl)-ethyl]-ureido}-3-(pyridin-3-ylmethoxy)-isothiazole-4-carboxylic acid amide;
5-(3-Phenethyl-ureido)-3-(pyridin-3-ylmethoxy)-isothiazole-4-carboxylic acid amide;
5-{3-[2-(3-Ethoxy-4-methoxy-phenyl)-ethyl]-ureido}-3-(pyridin-3-ylmethoxy)-isothiazole-4-carboxylic acid amide;
5-{3-[2-(4-Ethoxy-phenyl)-ethyl]-ureido}-3-(pyridin-3-ylmethoxy)-isothiazole-4-carboxylic acid amide;
5-{3-[2-(4-Chloro-phenyl)-ethyl]-ureido}-3-(pyridin-3-ylmethoxy)-isothiazole-4-carboxylic acid amide;
5-{3-[2-(3-Chloro-phenyl)-ethyl]-ureido}-3-(pyridin-3-ylmethoxy)-isothiazole-4-carboxylic acid amide;
5-{3-[2-(3-Methoxy-phenyl)-ethyl]-ureido}-3-(pyridin-3-ylmethoxy)-isothiazole-4-carboxylic acid amide;
5-{3-[2-(4-Methoxy-phenyl)-ethyl]-ureido}-3-(pyridin-3-ylmethoxy)-isothiazole-4-carboxylic acid amide;
5-{3-[2-(3-Bromo-4-methoxy-phenyl)-ethyl]-ureido}-3-(pyridin-3-ylmethoxy)-isothiazole-4-carboxylic acid amide;
5-{3-[2-(4-Bromo-phenyl)-ethyl]-ureido}-3-(pyridin-3-ylmethoxy)-isothiazole-4-carboxylic acid amide;
5-{3-[2-(2-Chloro-phenyl)-ethyl]-ureido}-3-(pyridin-3-ylmethoxy)-isothiazole-4-carboxylic acid amide;
5-{3-[2-(3-Chloro-phenyl)-ethyl]-ureido}-3-(pyridin-3-ylmethoxy)-isothiazole-4-carboxylic acid amide;
5-{3-[2-(2-Fluoro-phenyl)-ethyl]-ureido}-3-(pyridin-3-ylmethoxy)-isothiazole-4-carboxylic acid amide;
5-{3-[2-(3-Fluoro-phenyl)-ethyl]-ureido}-3-(pyridin-3-ylmethoxy)-isothiazole-4-carboxylic acid amide;
5-{3-[2-(4-Fluoro-phenyl)-ethyl]-ureido}-3-(pyridin-3-ylmethoxy)-isothiazole-4-carboxylic acid amide;
5-{3-[2-(4-Ethoxy-3-methoxy-phenyl)-ethyl]-ureido}-3-(pyridin-3-ylmethoxy)-isothiazole-4-carboxylic acid amide;
5-{3-[2-(3-Ethoxy-4-methoxy-phenyl)-ethyl]-ureido}-3-(pyridin-3-ylmethoxy)-isothiazole-4-carboxylic acid amide;
5-{3-[2-(2,5-Dimethoxy-phenyl)-ethyl]-ureido}-3-(pyridin-3-ylmethoxy)-isothiazole-4-carboxylic acid amide;
5-{3-[2-(3-Methoxy-phenyl)-ethyl]-ureido}-3-(pyridin-3-ylmethoxy)-isothiazole-4-carboxylic acid amide;
5-[3-(2-Difluoromethoxy-benzyl)-ureido]-3-(pyridin-3-ylmethoxy)-isothiazole-4-carboxylic acid amide;
5-[3-(2,6-Dimethoxy-benzyl)-ureido]-3-(pyridin-3-ylmethoxy)-isothiazole-4-carboxylic acid amide;
5-[3-(2,5-Dichloro-benzyl)-ureido]-3-(pyridin-3-ylmethoxy)-isothiazole-4-carboxylic acid amide;
5-[3-(3-Morpholin-4-yl-propyl)-ureido]-3-(pyridin-3-ylmethoxy)-isothiazole-4-carboxylic acid amide;
5-[3-(2-Morpholin-4-yl-ethyl)-ureido]-3-(pyridin-3-ylmethoxy)-isothiazole-4-carboxylic acid amide;
5-[3-(2-Diethylamino-ethyl)-ureido]-3-(pyridin-3-ylmethoxy)-isothiazole-4-carboxylic acid amide;
5-[3-(3-Dimethylamino-propyl)-ureido]-3-(pyridin-3-ylmethoxy)-isothiazole-4-carboxylic acid amide;
5-{3-[2-(1-Methyl-pyrrolidin-2-yl)-ethyl]-ureido}-3-(pyridin-3-ylmethoxy)-isothiazole-4-carboxylic acid amide;
5-{3-[3-(2-Methyl-piperidin-1-yl)-propyl]-ureido}-3-(pyridin-3-ylmethoxy)-isothiazole-4-carboxylic acid amide;
(R), (R)-5-[3-(2-Hydroxy-cycloheptylmethyl)-ureido]-3-(pyridin-3-ylmethoxy)-isothiazole-4-carboxylic acid amide;
(R),(R)-5-[3-(2-Hydroxy-cyclooctylmethyl)-ureido]-3-(pyridin-3-ylmethoxy)-isothiazole-4-carboxylic acid amide;
5-[3-(2-Hydroxy-ethyl)-ureido]-3-(pyridin-3-ylmethoxy)-isothiazole-4-carboxylic acid amide;
5-[3-(2-Hydroxy-butyl)-ureido]-3-(pyridin-3-ylmethoxy)-isothiazole-4-carboxylic acid amide;
5-{3-[3-(2—Oxo-pyrrolidin-1-yl)-propyl]-ureido}-3-(pyridin-3-ylmethoxy)-isothiazole-4-carboxylic acid amide;
5-[3-(3-Imidazol-1-yl-propyl)-ureido]-3-(pyridin-3-ylmethoxy)-isothiazole-4-carboxylic acid amide;
5-(3-Benzyl-ureido)-3-(pyridin-3-ylmethoxy)-isothiazole-4-carboxylic acid amide;
5-[3-(2,5-Difluoro-benzyl)-ureido]-3-(pyridin-3-ylmethoxy)-isothiazole-4-carboxylic acid amide;
3-(1-Pyridin-3-yl-ethoxy)-5-(3-pyridin-2-ylmethyl-ureido)-isothiazole-4-carboxylic acid amide;
5-[3-(2,6-Dimethoxy-benzyl)-ureido]-3-(1-pyridin-3-yl-ethoxy)-isothiazole-4-carboxylic acid amide;
5-(3-Cyclopropylmethyl-ureido)-3-(pyridin-3-ylmethoxy)-isothiazole-4-carboxylic acid amide;
5-(3-Methyl-ureido)-3-(pyridin-3-ylmethoxy)-isothiazole-4-carboxylic acid amide;
5-(3-Methyl-ureido)-3-(1-pyridin-3-yl-ethoxy)-isothiazole-4-carboxylic acid amide; and
5-[3-(3,5-Dichloro-benzyl)-ureido]-3-(pyridin-3-ylmethoxy)-isothiazole-4-carboxylic acid amide.

13. A pharmaceutical composition comprising a compound of any one of claims 1-12 and a pharmaceutically acceptable carrier.

14. A method of treating a TGF-related disease state in a mammal comprising the step of administering to the mammal suffering from the TGF-related disease state a therapeutically effective amount of a compound of any one of claims 1-12.

15. A method of claim 8, wherein said TGF-related disease state is selected from the group consisting of hyperproliferative disorders and fibrotic diseases.
